(19) United States
(12) Patent Application Publication
(10) Pub. No.: US 2010/0063531 A1
(43) Pub. Date: Mar. 11, 2010

(54) Medical Device with Non-Circumferential Surface Portion

(75) Inventors: Leon Rudakov, San Marcos, CA (US); Michael O'Connor, Singapore (SG); Vimal Raj D, Singapore (SG)

Correspondence Address: MCDERMOTT WILL & EMERY LLP, 18191 VON KARMAN AVE., SUITE 500, IRVINE, CA, 92612-7108, US

(73) Assignee: MERLIN MD PTE LTD., #04 NORTHTECH LOBBY 3 (SG)

(21) Appl. No.: 12/093,310

(22) PCT Filed: Nov. 9, 2005
(86) PCT No.: PCT/SG05/00383

§ 371 Date: Sep. 9, 2008

(51) Int. Cl. A61M 29/00; A61B 5/00

(52) U.S. Cl. 606/194; 600/309

(57) ABSTRACT

PCT A medical device (10) for insertion into a bodily vessel (4) to treat an aneurysm (5) having an aneurysm neck, the device (10) comprising: a mechanically expandable device expandable from a first position to a second position; the mechanically expandable device having an exterior circumferential surface at end portions (11, 12) of the mechanically expandable device such that the exterior circumferential surface engages with the inner surface of the vessel (4) so as to maintain a fluid pathway through said vessel (4) when the end portions (11, 12) of the mechanically expandable device are expanded radially outwardly to the second position; the mechanically expandable device having an exterior non-circumferential surface at a connecting portion (13) of the mechanically expandable device to connect the end portions (11, 12); and an expandable membrane (15) extending over a portion of the exterior non-circumferential surface, the membrane (15) is expanded in response to expansion of the mechanically expandable device; wherein the connecting portion (13) is positioned proximal to the aneurysm neck such that the expanded membrane (15) obstructs blood circulation to the aneurysm (5).

### TECHNICAL FIELD

The invention concerns a medical device for insertion into a bodily vessel to treat an aneurysm having an aneurysm neck.

### BACKGROUND OF THE INVENTION

In tortuous vessel paths, conventional stents and delivery systems lack adequate flexibility when treating aneurysms associated with hemorrhagic diseases. In some cases, a conventional stent may straighten a natural curvature of a bodily vessel once the stent is deployed. This increases the vessel injury score and may lead to restenosis or other adverse events.

If the aneurysm is a bifurcation or trifurcation aneurysm, a conventional stent typically obstructs natural blood circulation to a vessel path other than the bifurcation or trifurcation branches. Blood must pass through the struts and structure of the stent to circulate to such a vessel path.

Thrombosis occurs on some occasions after a stent is deployed within the vessel due to irritation of the endothelial lining of the vessel. Thrombosis can be mitigated by covering a stent with a drug (drug-eluting stents).

Other ways to treat aneurysms is the use of coiling. If an aneurysm possesses a wide neck, stenting in combination with coiling is required. This type of procedure suffers from significant surgery time, sometimes 4 to 5 hours, it is expensive, and it leaves coils in the aneurysm for the rest of patient's life. This type of procedure cannot be used to treat a wide class of aneurysms such as wide neck aneurysms, giant aneurysms, or Carotid Cavernous Fistula.

Therefore, there is a desire for a medical device which has increased flexibility for deployment within a tortuous vessel path, minimises obstruction of blood circulation when treating bifurcation or trifurcation aneurysms, and minimises thrombosis.

### SUMMARY OF THE INVENTION

In a first preferred aspect, there is provided a medical device for insertion into a bodily vessel to treat an aneurysm having an aneurysm neck, the device comprising:
a mechanically expandable device expandable from a first position to a second position;
the mechanically expandable device having an exterior circumferential surface at end portions of the mechanically expandable device such that the exterior circumferential surface engages with the inner surface of the vessel so as to maintain a fluid pathway through said vessel when the end portions of the mechanically expandable device are expanded radially outwardly to the second position;
the mechanically expandable device having an exterior non-circumferential surface at a connecting portion of the mechanically expandable device to connect the end portions; and
an expandable membrane extending over a portion of the exterior non-circumferential surface, the membrane is expanded in response to expansion of the mechanically expandable device;
wherein the connecting portion is positioned proximal to the aneurysm neck such that the expanded membrane obstructs blood circulation to the aneurysm.

The connecting portion may comprise a plurality of longitudinal members extending along an axis parallel to the longitudinal axis of the mechanically expandable device.

The longitudinal members may be interconnected by deformable linking members to ensure the device is not extended longitudinally beyond a predetermined longitudinal length.

The deformable linking members may be “C” shaped.

The membrane may extend along the entire exterior non-circumferential surface and a portion of the exterior circumferential surface of each of the end portions.

Each longitudinal member may comprise a series of: a first inclined section, a straight section and a second inclined section angled opposite to the first inclined section.

Radiopaque markers may be positioned at the distal ends of the device to enhance visualization and positioning of the device during deployment.

The connecting portion may be made from a radiopaque material, the radiopaque material being any one from the group consisting of: Platinum Iridium alloy and Platinum Tungsten alloy.

The medical device may be made from stainless steel or Nitinol.

In a second aspect, there is provided a delivery system for delivering the medical device as described, the system comprising:
an inflatable member to expand the medical device from the first position to the second position;
a rotatable system to rotate the medical device in the bodily vessel; and
an aneurysm detection member to detect the location of the aneurysm relative to the medical device;
wherein the rotatable system and aneurysm detection member ensure the connecting portion is positioned proximal to the aneurysm neck when the medical device is expanded such that the expanded membrane obstructs blood circulation to the aneurysm.

The inflatable member may be a train balloon or asymmetric balloon. Using these types of balloons in a delivery system enhances system flexibility and significantly reduces or eliminates the problem of vessel straightening during deployment.

The train balloon may comprise a plurality of balloons that are interlinked by a bridging portion, each balloon expanding each end portion of the medical device upon inflation.

The bridging portion may be formed by applying a restriction ring to physically constrain the train balloon at bridging portion.

The asymmetric balloon may comprise balloon end portions connected by a relatively smaller central portion, each balloon end portion expanding each end portion of the medical device upon inflation.

The rotatable system may be a monorail balloon system or pull wire rotation system.

The monorail balloon system may comprise a first shaft in mating relationship with a second shaft extending from the inflatable member, and movement of the first shaft along the longitudinal axis of the first shaft relative to the second shaft causes the inflatable member to rotate and the medical device to rotate in the bodily vessel.

The pull wire rotation system may comprise a first shaft in mating relationship with a second shaft extending from the inflatable member, and a wire wound around the circumferential surface of the second shaft and secured to the first shaft, and movement of the first shaft along the longitudinal axis of the first shaft in a direction away from the second shaft causes the inflatable member to rotate and the medical device to rotate in the bodily vessel.

The aneurysm detection member may be any one from the group consisting of: optical sensor, radiopaque antenna head, and intravascular ultrasound (IVUS).

The optical sensor may transmit and receive light directed towards the aneurysm, and the location of the aneurysm relative to the medical device is determined if a difference in light level is sensed.

The radiopaque antenna head may be movable from a retracted position to an extended position, and the location of the aneurysm relative to the medical device is determined if the radiopaque antenna head enters within the aneurysm.

In a third aspect, there is provided a delivery system for delivering a medical device to a surgical site in a bodily vessel to treat an aneurysm, the system comprising:
an inflatable member to expand the medical device from a first position to a second position, the mechanically expandable device is expanded radially outwardly to the second position;
a rotatable system to rotate the medical device in the bodily vessel; and
an aneurysm detection member to detect the location of the aneurysm relative to the medical device;
wherein the rotatable system and aneurysm detection member ensure the connecting portion is positioned proximal to the aneurysm neck when the medical device is expanded such that the expanded membrane obstructs blood circulation to the aneurysm.

The aneurysm may be a bifurcation or trifurcation aneurysm.

In a fourth aspect, there is provided a method for deploying the medical device as described, the method comprising:
supplying a first amount of an inflation medium via a balloon catheter to partially inflate a balloon and cause the end portions to expand to a first predetermined diameter;
adjusting the orientation and position of the medical device by rotating the balloon such that the membrane is positioned proximal to the aneurysm neck; and
supplying a second amount of an inflation medium via a balloon catheter to fully inflate the balloon and cause the end portions to expand to a second predetermined diameter such that the expanded membrane obstructs blood circulation to the aneurysm.

The balloon may be a train balloon or asymmetric balloon.

The first predetermined diameter may be about 1.5 to 2.0 mm.

The second predetermined diameter may be about 2.5, 3.0 or 4.0 mm.

### BRIEF DESCRIPTION OF THE DRAWINGS

An example of the invention will now be described with reference to the accompanying drawings, in which:

FIG. 1 is a schematic diagram of a medical device in accordance with a preferred embodiment of the present invention deployed in a bodily vessel to obstruct blood circulation to an aneurysm;

FIG. 2 is a side view of the medical device of FIG. 1;

FIG. 3 is a perspective view of the medical device of FIG. 1 where a membrane is positioned above a connecting portion of the medical device;

FIG. 4 is a perspective view of the medical device of FIG. 1 where a membrane is positioned beneath a connecting portion of the medical device;

FIG. 5 is a side view of the medical device of FIG. 1 expanded by a train balloon catheter;

FIG. 6 are a series of views of the train balloon catheter of FIG. 5;

FIG. 7 are a series of views of an asymmetric balloon to expand the medical device of FIG. 1;

FIG. 8 is a perspective view of a monorail system to rotate a train balloon during expansion of the medical device of FIG. 1;

FIG. 9 is a side view of a monorail system to rotate an asymmetric balloon during expansion of the medical device of FIG. 1;

FIGS. 10 to 13 are side views of a pull wire rotation system to rotate a train balloon or asymmetric balloon during expansion of the medical device of FIG. 1;

FIG. 14 is a side view of an optical sensor to determine the position of the aneurysm relative to the medical device;

FIG. 15 is a side view of radiopaque antenna head to determine the position of the aneurysm relative to the medical device;

FIG. 16 is an exploded side view of the medical device of FIG. 1 expanded by a train balloon catheter; and

FIGS. 17 and 18 are views of a rail lumen to deliver the medical device to a surgical site.

### DETAILED DESCRIPTION OF THE DRAWINGS

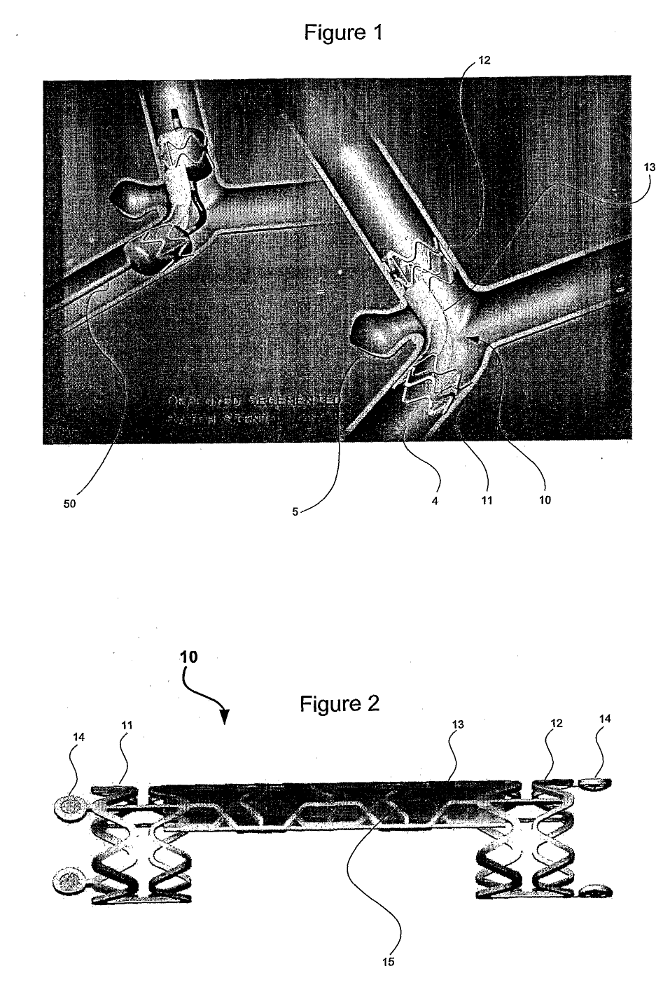

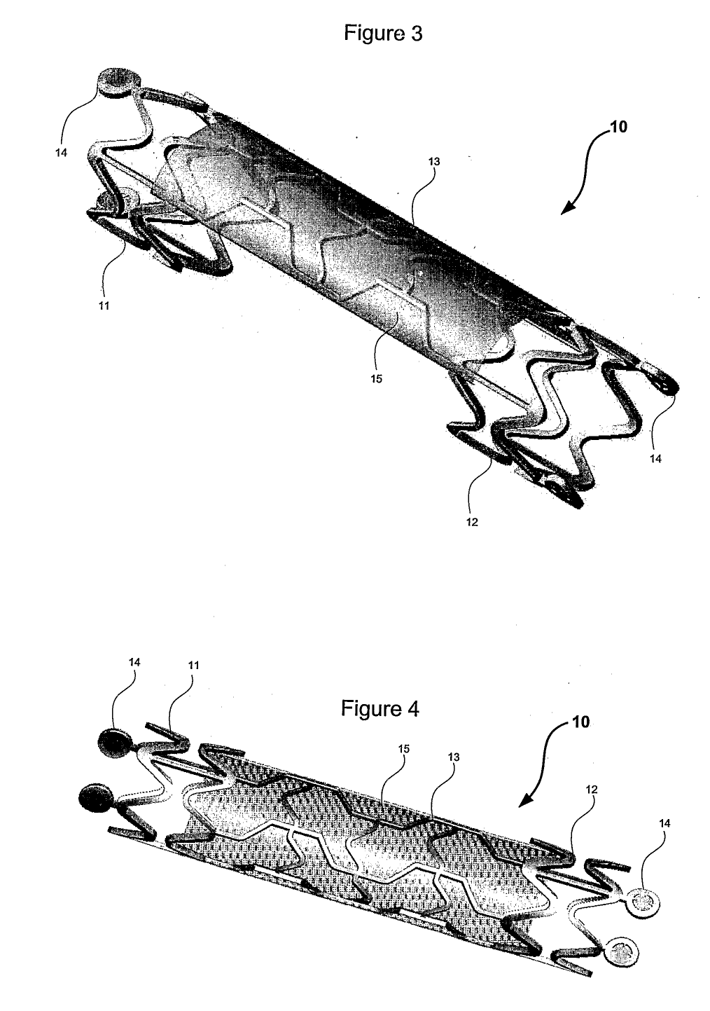

Referring to FIGS. 1 to 4, there is provided a medical device 10 for insertion into a bodily vessel 4 to treat an aneurysm 5 having an aneurysm neck. The aneurysm 5 may be associated with hemorrhagic diseases. The device 10 comprises: a stent portion and an expandable membrane 15. The stent portion is expandable from a first position to a second position. The stent portion is expanded radially outwardly to the second position. The stent portion has an exterior circumferential surface at end portions 11, 12 of the stent such that the exterior circumferential surface engages with the inner surface of the vessel 4 so as to maintain a fluid pathway through said vessel 4 when the stent portion is expanded to the second position. The stent portion has an exterior non-circumferential surface at a connecting portion 13 of the stent to connect the end portions 11, 12. The expandable membrane 15 extends over a portion of the exterior non-circumferential surface. The membrane 15 is expanded in response to expansion of the stent portion. The connecting portion 13 is positioned proximal to the aneurysm neck such that the expanded membrane 15 obstructs blood circulation to the aneurysm 5. One advantage of the medical device 10 is that there is minimal surface contact between the struts and membrane 15 of the medical device 10 and the vessel wall 4 thereby reducing thrombosis. The membrane 15 is intended to only cover and seal the aneurysm neck and does not cover other areas of the vessel 4.

A biological advantage of the medical device 10 is that it causes significantly less thrombosis compared to a conventional stent with a membrane 15 wrapped around the exterior circumferential surface of the stent. One reason for this is that the contact surface area between the vessel 4 and the device 10 is reduced because the device 10 has a reduced exterior circumferential surface. Only end portions 11, 12 of the stent portion form the exterior circumferential surface of the medical device 10 whereas the connecting portion 13 is an exterior non-circumferential surface of the stent joining the end portions 11, 12 together. Further, there is minimal risk to blood vessel perforators since the membrane 15 is positioned only at the neck of the aneurysm 5 leaving the rest of the vessel 4 undisturbed and unobstructed to blood circulation.

The end portions 11, 12 of the medical device 10 are constructed by circumferential struts. The circumferential struts at the end portions 11, 12 of the medical device 10 are responsible for the opening of the stent portion. The end struts are connected by longitudinal struts. The longitudinal struts are interconnected by C-interlink struts, oriented generally transverse to the longitudinal struts.

The connecting portion 13 is comprises three horizontal zigzag struts with interconnected by C-interlink struts. The connecting portion 15 opens freely with minimal resistance together with the membrane 15 when the balloon is expanded.

The zigzag design increases flexibility of the medical device 10 at the connecting portion 15 and the C-interlink struts provide structural integrity for the connecting portion 13 and membrane 15 when they are expanded. The design also ensures that the overall length of the medical device 10 does not vary beyond a predefined range.

The connecting portion 13 may be made from stainless steel. The entire medical device 10 is cut from stainless steel including the connecting portion 13. Alternatively, the connecting portion 13 may be made from Platinum-Iridium or Platinum-Tungsten. Pt—Ir/Pt—W causes the entire connecting portion 13 to be radiopaque which makes it easier for positioning and alignment of the membrane 15 in the vessel 4 relative to the aneurysm 5.

In another embodiment, the connecting portion 13 may be made from Nitinol. In this embodiment, the medical device 10 is balloon expandable with a self-expanding connecting portion 13 made from Nitinol. Markers 14 are also used to assist in visualization and positioning of the membrane 15 in the vessel 4 and relative to the aneurysm 5.

The membrane 15 is aligned with respect to the connecting portion 13. The membrane 15 may be positioned above or beneath the struts of the connecting portion 13, depending on usage. In one embodiment, the membrane 15 is placed over the connecting portion 13 of the medical device 10. When the end portions 11, 12 are expanded, the connecting portion 13 expands also. The longitudinal struts and the C-interlink struts of the connecting portion 13 provide support for the membrane 15, similar to a scaffold.

The medical device 10 may be entirely of a single material, for example, stainless steel or Nitinol, or may be made from a combination of different materials Alternatively, the connecting portion 13 may be fabricated separately from and later attached to the end struts of the end portions 11, 12. The connecting portion 13 may be prefabricated from Platinum-Iridium or Platinum-Tungsten alloy. There are several ways to attach the connecting portion 13 to the end portions 11, 12 in order to construct the medical device 10. If the connecting portion 13 is prefabricated using a different material (Platinum-tungsten) to the end portions 11, 12 (stainless steel), forging or welding may be used. Mechanical forging for joining contact surfaces located at the ends of longitudinal struts and the end struts may be used. Alternatively, the contact surfaces may be laser spot welded together.

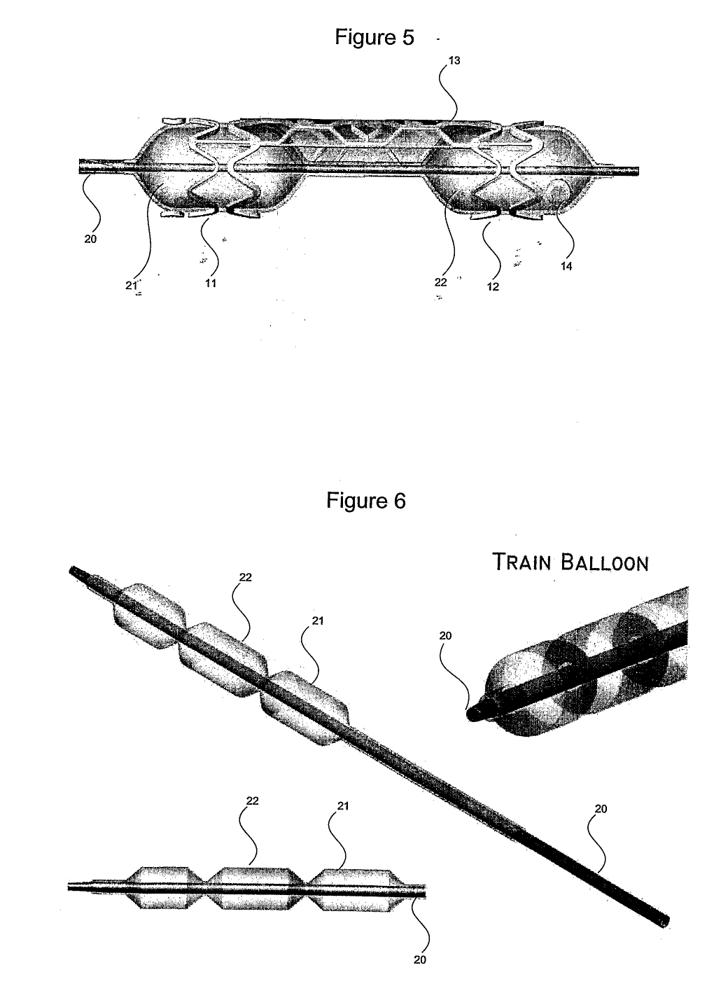

Referring to FIG. 5, the medical device 10 is balloon expandable. When the balloon (train balloon 20 or asymmetric 30) is expanded, the end struts at the end portions 11, 12 expand causing the connecting portion 13 to expand also. The membrane 15 also expands, similar to an umbrella when it is opened. Alternatively, Nitinol may be used to make the medical device 10 self-expandable or to assist in the deployment process where the medical device 10 is balloon expandable.

Radiopaque markers 14 for visualization and positioning of the medical device 10 may be included. Gold/platinum or other radiopaque markers for visualization may be used.

### Delivery System

A delivery system for tracking, aligning and deploying the medical device 10 is provided. Due to the novel structure of the medical device 10, a delivery system that is capable of delivering the medical device 10 through tortuous vessel paths, for example, in the intracranial region, is required.

As described earlier, the medical device 10 allows for a balloon to expand the end struts of the end portions 11, 12. When the end struts are expanded by the balloon, the connecting portion 13 together with the membrane 15 expands.

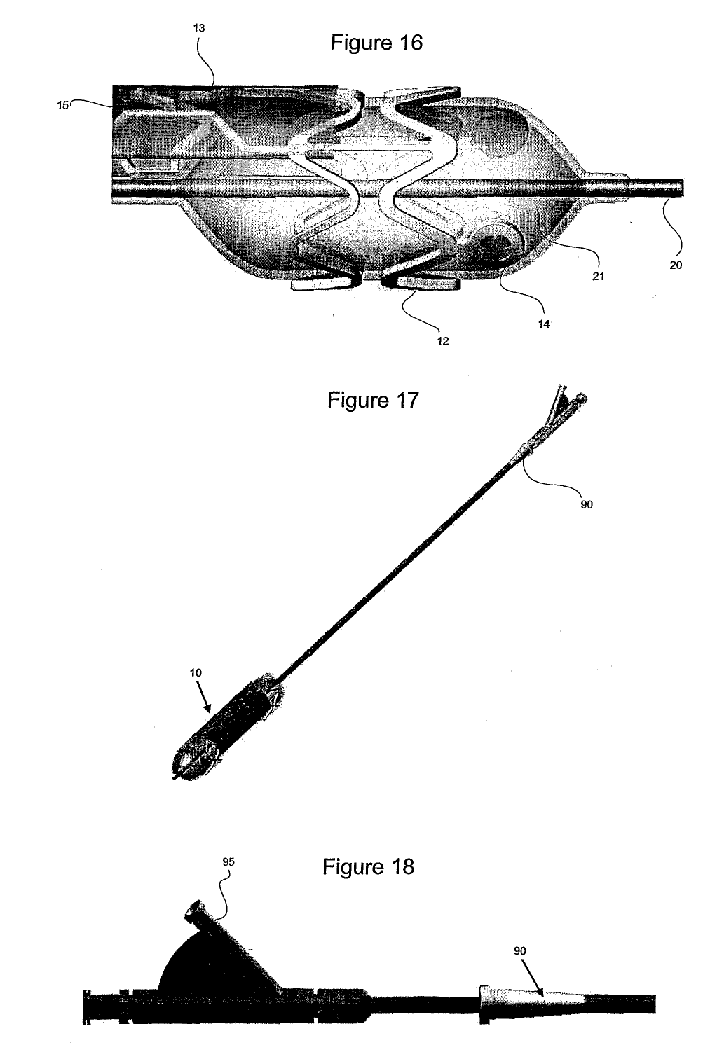

Referring to FIGS. 5, 16, 17 and 18, a train balloon catheter 20, 50 is used to expand the end struts of the end portions 11, 12. The balloon portion 21, 22 of the balloon catheter 20 is segmented into two or more sections connected by bridge portions. In response to actuation, a trigger 95 compresses air through a nozzle 90 and via the catheter 20 to inflate the balloon portions 21, 22.

At least two short balloon portions 21, 22 are connected by the interlinking bridge portions. The inner lumen of the balloon catheter 20 (guide wire lumen) passes through the centre of the balloons 21, 22. The outer lumen of the balloon catheter 20 delivers the inflation medium and is designed such that all the balloon portions 21, 22 open simultaneously when the inflation medium is applied. Inflation of the balloon portions 21, 22 at substantially the same time is achieved by controlling the inner diameter of the outer lumen at the bridge portions. From the source side of the inflation medium, a larger inner diameter may be used for distal balloon portions, and a smaller inner diameter may be used for proximal balloon portions. A predetermined amount of inflation medium is ensured to reach each balloon portion 21, 22.

Using a train balloon 20 reduces the injury score to the vessel 4 due to minimum surface area contact with vessel 4. That is, less balloon surface makes contact with the vessel wall. The train balloon 20 also has a highly flexible distal section, and does not straighten a tortuous vessel during expansion.

Train balloons 20 with shorter bridge portions may also be used with conventional stents, that is, stents without a connecting portion 13 or membrane 15. A train balloon 20 is highly effective for stenting a tortuous vessel without compromising the shape of the vessel. In contrast to conventional balloons, the train balloon 20 curves with the natural curvature of a vessel during expansion.

The balloon portions 21, 22 can be extruded separately with a smaller bridge portion and joined together. Alternatively, the train balloon 20 may be fabricated using a long regular semi-compliant or compliant balloon and restriction rings are applied to form the bridge portions due to constraining the semi/compliant balloon. The restriction rings physically restrict the expansion of the train balloon at the site of the restriction rings to ensure that the train balloon 20 is not expanded at these regions making them the flexible points during expansion. The restriction rings are secured to the balloon 20, for example, by adhesive or thermally bonded.

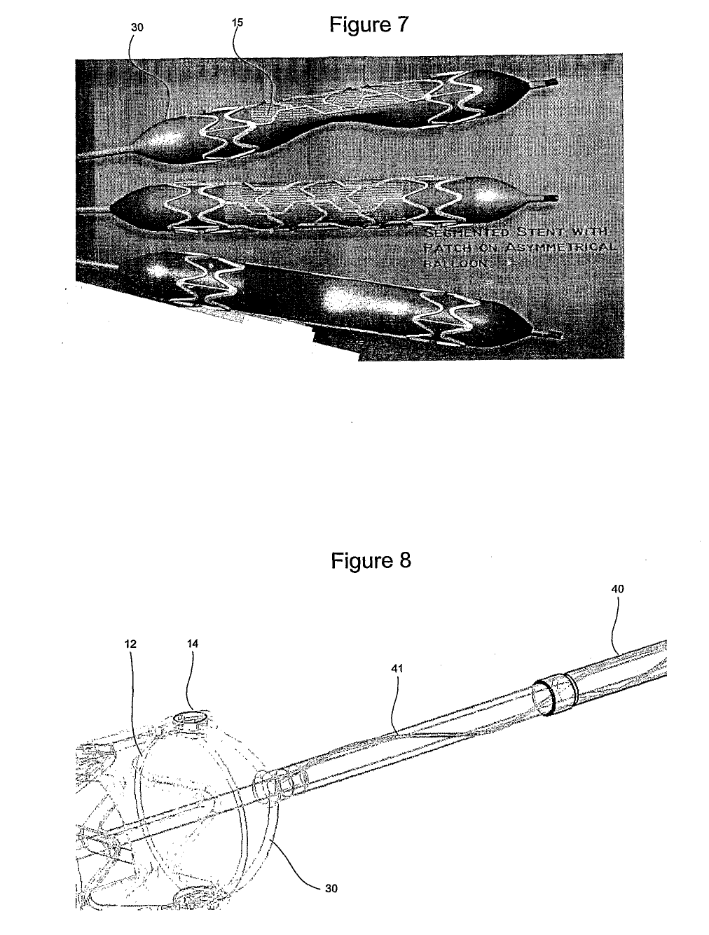

Referring to FIG. 7, in another embodiment, instead of a train balloon 20, an asymmetric balloon 30 is used to inflate the end portions 11, 12. The balloon 30 is extruded to form a crescent-like cross section in the middle and circular-shaped portions at the ends of the balloon 30. As with the train balloon 20, the central portion of the balloon 30 is constrained, similar to the bridge portions of the train balloon 20. This restricts contact to only the connecting portion 13 with the central portion of the balloon exterior surface.

A balloon rotating mechanism is provided for rotating of the balloon 20, 30 in order to rotate the medical device 10 and position the connecting portion 13 and membrane 15 against the aneurysm neck to obstruct blood circulation to the aneurysm 5. The balloon catheter 20, 30 has a rotatable distal section which is activated during deployment. Once the position of the aneurysm 5 is determined, the distal section is rotated from the proximal end of the balloon catheter 20, 30. A two step inflation process is used. Initially, the end portions 11, 12 are half expanded when the medical device 10 reaches the site of the aneurysm 5. Next, the position and orientation of the membrane 15 is slightly adjusted, if required, and then the end portions 11, 12 are completely expanded to deploy the medical device 10. In this two stage inflation process, the balloon is inflated to expand the end portions 11, 12 up to 1.5 to 2.0 mm diameter for the first stage of deployment. This allows additional minor adjustment to the orientation of the membrane 15 towards the direction of aneurysm neck. The second stage of deployment is the post orientation stage where the membrane 15 is comfortably positioned against the aneurysm neck. At the second stage, the medical device 10 is then expanded to its nominal diameter 2.5, 3.0 or 4.0 mm. Thus, the aneurysm neck is effectively sealed from the blood circulation in the vessel 4.

Two possible mechanisms for rotating the balloon 20, 30 are described: a monorail balloon system and a pull wire rotation system. It is envisaged that other mechanisms for rotating the balloon are possible.

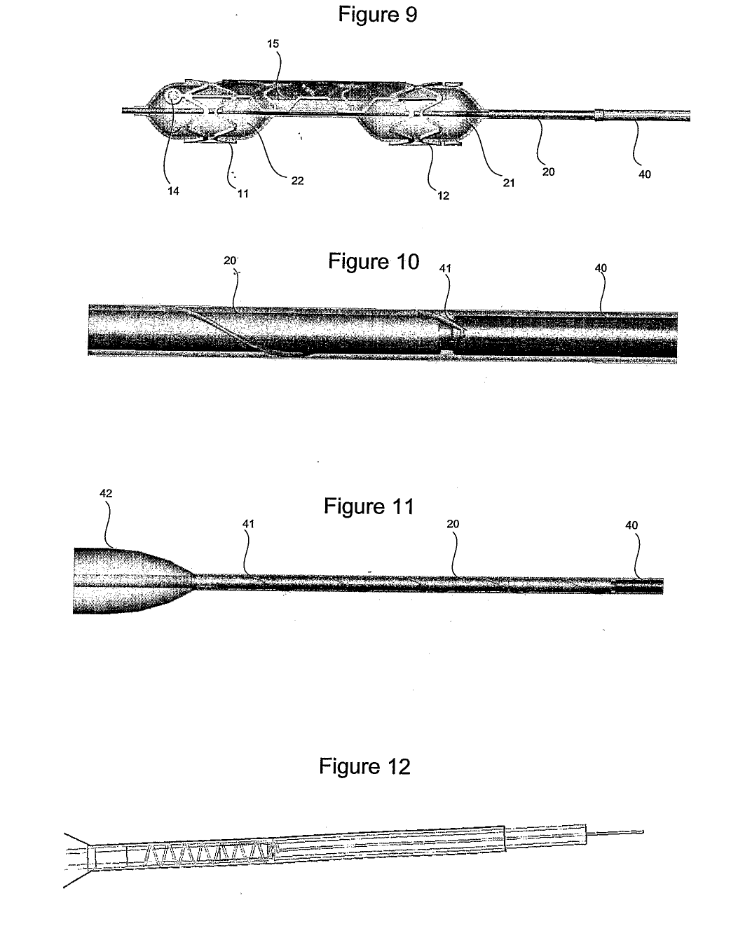

Referring to FIGS. 8 and 9, the monorail balloon system enables rotation of the balloon distal section when deploying the medical device 10. The distal section of the balloon catheter 20, 30 has a threaded groove 41 on the exterior surface of the distal section. The groove 41 is approximately 10 to 15 cm in length from the proximal balloon bond. The balloon catheter is used with a micro-catheter 40 or a third lumen (rail lumen). The distal end of the micro-catheter 40 has a groove on its interior surface in mating relationship with the groove 41 of the balloon catheter. The distal section is rotated by pushing or pulling the micro-catheter 40 relative to the balloon catheter. In an alternate embodiment, the grooves may be swapped from the interior to exterior surface.

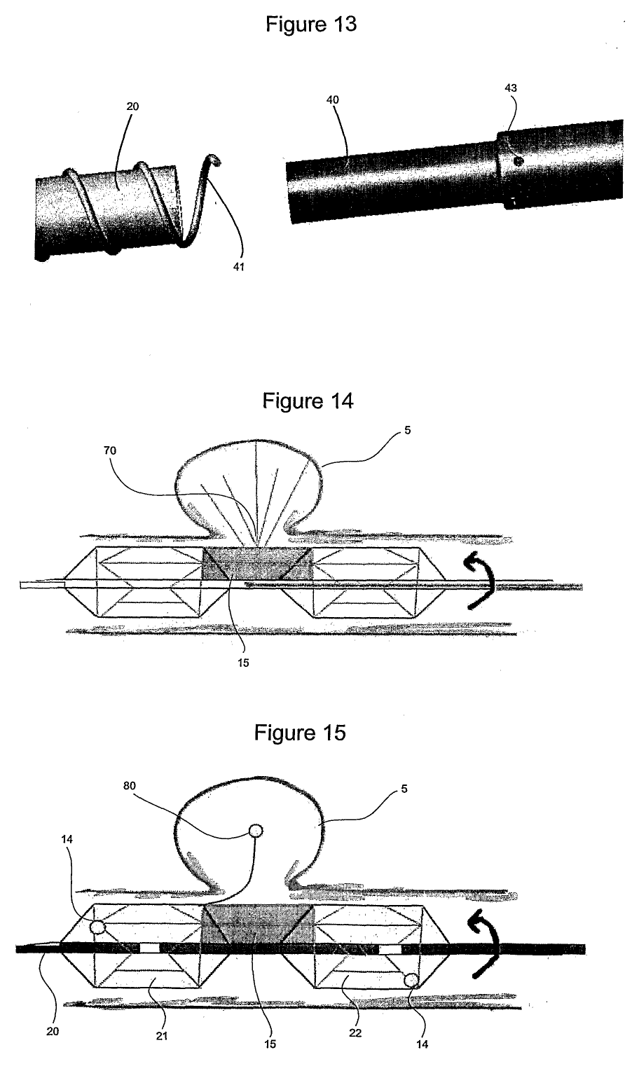

Referring to FIGS. 10 to 13, the pull wire rotation system enables rotation of the balloon distal section by pulling a thin pull wire 41 that is wound around a rotatable distal balloon section 20. The distal section 20 continues to the distal soft tip of the balloon 21. The balloon 21 is joined to the rotatable section 20 at the distal and proximal balloon end. The proximal end of the balloon 21 forms a rotatable joint with the inner lumen 40. The distal end of the pull wire 41 is attached to the rotatable section 20, then coils around a groove on the rotatable section 20, enters the inner lumen 40 via an aperture 43 on the distal part and exits via the hub 42 at the proximal end of the pull wire rotation system.

The rotatable section 20 forms a rotary joint between the distal end of the inner lumen 40 and the distal balloon section 20. The rotary joint may be lubricated to ease rotation of both parts. When the pull wire 41 is pulled from the proximal end, the coiled portion of the wire 41 on the rotatable section 20 start to unwind, which causes rotation of the rotatable section 20. Rotation of the rotatable section 20 rotates the balloon 21.

If there is a failure to position the membrane 15 against the aneurysm neck on the first attempt, the Dull wire 41 may be continuously pulled for another revolution of the rotatable section 20 to properly align the membrane 15 on the next attempt. The rotary joint is pressure sealed to avoid leakage of the inflation medium. Capping the hub 42 is one example to ensure a pressure seal.

In order to deploy the medical device 10 at the correct position, the location of the aneurysm 5 in the bodily vessel 4 must be determined. This may be achieved by: using optical sensor technology 70, a radiopaque antenna head 80 to locate the aneurysm 5 physically using a radiopaque wire or using ultra sound technology (for example, IVUS).

Referring to FIG. 14, optical sensor technology such as optical fiber cable 70 may be used to transmit and receive light from the site of the aneurysm 5. The proximal end of the optical fiber 70 is connected to a sensor which converts the light signals to electrical signals. The electrical signals are displayed using a real time visualization system. The optical fiber 70 is rotated at the site of the aneurysm 5 until a difference in signal levels is detected when light enters the aneurysm 5. Once the position of the aneurysm 5 is determined, the balloon is expanded and the medical device 10 is deployed

Referring to FIG. 15, a radio-opaque antenna head 80 connected by a wire anchored to the medical device 10 is used to locate the aneurysm 5 physically. The antenna 80 is mounted above the membrane 15 longitudinally along the connecting portion 15 during manufacture of the medical device 10. The antenna 80 may be made from a shape memory material (for example, Nitinol) and physically restricted from releasing until the balloon reaches the site of the aneurysm 5. Marker bands 14 on the balloon and the markers 14 on the medical device 10 assist in positioning the connecting portion 15 across the aneurysm neck. Next, the physical restriction is removed from the antennae 80 when the balloon 21, 22 is partially expanded. Once the antenna 80 is released, it springs up due to the shape memory and slightly pushes against the vessel wall. The pressure exerted by the antenna 80 is low so as to not cause any injury to the vessel 4.

The balloon catheter is rotated until the antenna head 80 finds the aneurysm neck and enters within the aneurysm 5. The radiopaque head 80 within the aneurysm 5 clearly confirms the alignment of the membrane 15 to the aneurysm neck using angiography. Next, the balloon is completely expanded to cause deployment of the medical device 10. The antenna 80 remains mounted to the medical device 10 and remains within the aneurysm 5. The balloon catheter is subsequently removed from the vessel 4.

Intravascular ultrasound (IVUS) technology may be used combination with the medical device 10 to locate and deploy the medical device 10 at the site of the aneurysm 5. IVUS is a medical imaging methodology which uses specially designed catheters attached to computerized ultrasound equipment. IVUS uses ultrasound technology to visualize the inner wall of blood vessels from inside a blood vessel out through the surrounding blood column. IVUS is used in particular for the anatomy of the walls of blood vessels including aneurysms.

In IVUS, an ultrasound catheter tip is slid over a guide wire. The ultrasound catheter tip is positioned using angiography techniques so that the tip is at the most distal position to be imaged. The sound waves are emitted from the catheter tip, and are usually in the 10 to 20 MHz range. The catheter also receives and conducts the return echo information to an external computerized ultrasound equipment which constructs and displays a real time ultrasound image of a thin section of the blood vessel currently surrounding the catheter tip, usually displayed at approximately 30 frames per second.

It will be appreciated by persons skilled in the art that numerous variations and/or modifications may be made to the invention as shown in the specific embodiments without departing from the scope or spirit of the invention as broadly described. The present embodiments are, therefore, to be considered in all respects illustrative and not restrictive.

We claim:

1. A medical device for insertion into a bodily vessel to treat an aneurysm having an aneurysm neck, the device comprising:
a mechanically expandable device expandable from a first position to a second position;
the mechanically expandable device having an exterior circumferential surface at end portions of the mechanically expandable device such that the exterior circumferential surface engages with the inner surface of the vessel so as to maintain a fluid pathway through said vessel when the end portions of the
mechanically expandable device are expanded radially outwardly to the second position;
the mechanically expandable device having an exterior non-circumferential surface at a connecting portion of the mechanically expandable device to connect the end portions; and
an expandable membrane extending over a portion of the exterior non-circumferential surface, the membrane is expanded in response to expansion of the mechanically expandable device;
wherein the connecting portion is positioned proximal to the aneurysm neck such that the expanded membrane obstructs blood circulation to the aneurysm.

2. The device according to claim 1, wherein the connecting portion comprises a plurality of longitudinal members extending along an axis parallel to the longitudinal axis of the mechanically expandable device.

3. The device according to claim 2, wherein the longitudinal members are interconnected by deformable linking members to ensure the device is not extended longitudinally beyond a predetermined longitudinal length.

4. The device according to claim 3, wherein the deformable linking members are “C” shaped.

5. The device according to claim 1, wherein the membrane extends along the entire exterior non-circumferential surface and a portion of the exterior circumferential surface of each of the end portions.

6. The device according to claim 2, wherein each longitudinal member comprises a series of: a first inclined section, a straight section and a second inclined section angled opposite to the first inclined section.

7. The device according to claim 1, wherein radiopaque markers are positioned at the distal ends of the device to enhance visualization and positioning of the device during deployment.

8. The device according to claim 1, wherein the connecting portion is made from a radiopaque material, the radiopaque material being any one from the group consisting of: Platinum Iridium alloy and Platinum Tungsten alloy.

9. The device according to claim 1, wherein the device is made from stainless steel or Nitinol.

10. A delivery system for delivering the medical device according to claim 1, the system comprising:
an inflatable member to expand the medical device from the first position to the second position;
a rotatable system to rotate the medical device in the bodily vessel; and
an aneurysm detection member to detect the location of the aneurysm relative to the medical device;
wherein the rotatable system and aneurysm detection member ensure the connecting portion is positioned proximal to the aneurysm neck when the medical device is expanded such that the expanded membrane obstructs blood circulation to the aneurysm.

11. The system according to claim 10, wherein the inflatable member is a train balloon or asymmetric balloon.

12. The system according to claim 11, wherein the train balloon comprises a plurality of balloons that are interlinked by a bridging portion, each balloon expanding each end portion of the medical device upon inflation.

13. The system according to claim 12, wherein the bridging portion is formed by applying a restriction ring to physically constrain the train balloon at bridging portion.

14. The system according to claim 11, wherein the asymmetric balloon comprises balloon end portions connected by a relatively smaller central portion, each balloon end portion expanding each end portion of the medical device upon inflation.

15. The system according to claim 10, wherein the rotatable system is a monorail balloon system or pull wire rotation system.

16. The system according to claim 15, wherein the monorail balloon system comprises a first shaft in mating relationship with a second shaft extending from the inflatable member, and movement of the first shaft along the longitudinal axis of the first shaft relative to the second shaft causes the inflatable member to rotate and the medical device to rotate in the bodily vessel.

17. The system according to claim 15, wherein the pull wire rotation system comprises a first shaft in mating relationship with a second shaft extending from the inflatable member, and a wire wound around the circumferential surface of the second shaft and secured to the first shaft, and movement of the first shaft along the longitudinal axis of the first shaft in a direction away from the second shaft causes the inflatable member to rotate and the medical device to rotate in the bodily vessel.

18. The system according to claim 10, wherein the aneurysm detection member is any one from the group consisting of: optical sensor, radiopaque antenna head, and intravascular ultrasound (IVUS).

19. The system according to claim 18, wherein the optical sensor transmits and receives light directed towards the aneurysm, and the location of the aneurysm relative to the medical device is determined if a difference in light level is sensed.

20. The system according to claim 18, wherein the radiopaque antenna head is movable from a retracted position to an extended position, and the location of the aneurysm relative to the medical device is determined if the radiopaque antenna head enters within the aneurysm.

21. A delivery system for delivering a medical device to a surgical site in a bodily vessel to treat an aneurysm, the system comprising:
an inflatable member to expand the medical device from a first position to a second position, the mechanically expandable device is expanded radially outwardly to the second position;
a rotatable system to rotate the medical device in the bodily vessel; and
an aneurysm detection member to detect the location of the aneurysm relative to the medical device;
wherein the rotatable system and aneurysm detection member ensure the connecting portion is positioned proximal to the aneurysm neck when the medical device is expanded such that the expanded membrane obstructs blood circulation to the aneurysm.

22. The system according to claim 21, wherein the aneurysm is a bifurcation or trifurcation aneurysm.

23. A method for deploying the medical device according to claim 1, the method comprising:
supplying a first amount of an inflation medium via a balloon catheter to partially inflate a balloon and cause the end portions to expand to a first predetermined diameter;
adjusting the orientation and position of the medical device by rotating the balloon such that the membrane is positioned proximal to the aneurysm neck; and
supplying a second amount of an inflation medium via a balloon catheter to fully inflate the balloon and cause the end portions to expand to a second predetermined diameter such that the expanded membrane obstructs blood circulation to the aneurysm.

24. The method according to claim 23, wherein the balloon is a train balloon or asymmetric balloon.

25. The method according to claim 23, wherein the first predetermined diameter is about 1.5 to 2.0 mm.

26. The method according to claim 23, wherein the second predetermined diameter is about 2.5, 3.0 or 4.0 mm.
